(19) United States
(12) Patent Application Publication
(10) Pub. No.: US 2017/0143948 A1
(43) Pub. Date: May 25, 2017

(54) DEVICE FOR TREATING CUTANEOUS BLEMISHES AND DERMATOLOGIC DISEASES

(71) Applicant: FLAEM NUOVA S.P.A., Desenzano del Garda (BS) (IT)

(72) Inventors: Riccardo ABATE, Desenzano del Garda (BS) (IT); Roberto ALBERTELLI, Desenzano del Garda (BS) (IT); Mauro BERTELLI, Desenzano del Garda (BS) (IT)

(73) Assignee: FLAEM NUOVA S.P.A., Desenzano del Garda (BS) (IT)

(21) Appl. No.: 15/319,183

(22) PCT Filed: Jun. 19, 2015
(86) PCT No.: PCT/IB2015/054616

§ 371 Date: Dec. 15, 2016

(30) Foreign Application Priority Data
Jun. 24, 2014 (IT) BS2014A000117

(51) Int. Cl. A61M 35/00; A61M 1/08

(52) U.S. Cl. CPC A61M 35/00; A61M 1/08; A61M 2202/0208; A61M 2210/04; A61M 2205/505

(57) ABSTRACT

A device for treating cutaneous blemishes and dermatologic diseases that includes an application bell operatively connected to a feeding assembly is described. The bell is shaped to be removably leant on a cutaneous surface to be treated and with which it defines a treatment chamber. The feeding assembly includes, in its turn, a pump which realizes—in the treatment chamber—an environment having a pressure lower than the atmospheric pressure, and an oxygen delivering device activated subsequently to the pump, to deliver—in the treatment chamber—an oxygen flow. The feeding assembly provides for a controlling unit to selectively activate, in time sequence, the pump and the oxygen delivering device.

### FIELD OF THE INVENTION

The present invention relates to a device for aesthetic treatment used, in particular, but not exclusively, for the correction of cutaneous blemishes and dermatologic diseases.

### STATE OF THE ART

The hyperbaric chamber used in the treatment of chronic infections and torpid injuries of difficult healing, such as osteomyelites, fistulas, cutaneous ulcers, etc., is known.

In this type of treatment—commonly referred to as “district hyperbaric therapy”—the patient is locally treated with hyperbaric oxygen at a pressure higher than the atmospheric pressure. This causes a cutaneous revitalization and supports the transcutaneous penetration of active principles that may have been applied.

CN 102225224 describes an equipment for treating wounds that substantially follows the principle of district treatment in a hyperbaric chamber. In particular, the machine comprises an application bell intended to be positioned in abutment against the skin of the patient, next to a wound, and connected to a feeding circuit allowing to lead and keep the treatment area covered by the bell, in condition of negative pressure, and to supply oxygen to such an area.

The feeding circuit is configured for:

maintaining a substantially constant treatment pressure, lower than the atmospheric pressure, within the chamber defined by the bell and skin of the patient, and

feeding hyperbaric oxygen into the chamber.

More particularly, the feeding circuit comprises three main branches fluidically connected one with the others and connected to the bell. One first branch comprises a first vacuum pump to generate depression in the bell; one second branch comprises an oxygen generator, and a third branch comprises a second feeding pump to maintain a constant pressure in the treatment chamber. The vacuum pump is selectively activated with respect to the oxygen generator and the second feeding pump.

The device described above is however aimed at an exclusively medical field for the care of injuries or bruises on the cutis, or ulcers. According to the working principle, subjecting a wound in an environment having negative pressure in the presence of oxygen, aids the extraction of body fluids produced by the wound itself, in such a manner to support the healing. Instead, the same device cannot be applied for the treatment of skin blemishes.

The same application in medical field is however hardly appropriate. In fact, maintaining a wound in an environment having negative pressure and delivering hyperbaric oxygen at a pressure of about 1 atm, thus substantially high, as described in the same CN 102225224, causes various drawbacks.

Firstly, the administration of oxygen at high pressure doesn't aid the cicatrization of the wound, but on the contrary leads to its dilatation. The oxygen flow hitting the tissues around the wound causes dilation and the mutual spacing.

In particular, using the device on open wounds may involve bleeding, thereby prolonging the cicatrization time; it follows that it would be dangerous to use on recent wounds or sutures.

Secondly, from the operative point of view, the activation of the second pump in order to maintain the constant depression in the chamber of the bell, causes the outside air to mix with the oxygen thereby canceling the antibacterial properties of the hyperbaric oxygen itself. In other words, this causes a decrease of the oxygen concentration and thus a decrease of the treatment efficacy.

Thirdly, the duration of the process is long and hardly comfortable for the patient, indeed providing for several minutes of treatment both in the suction step and oxygenation step.

Document US 2004/260210 describes (FIGS. 14 and 23A) a device for treating cutaneous blemishes and dermatologic diseases (paragraphs 257, 288-290, 301, 333-336 and 387-405) comprising:

an application bell (10, 12, par. 259) shaped to be removably leant on a cutaneous surface to be treated and with which it defines a treatment chamber (par. 260 and par. 263);

a feeding assembly (70) operatively connected to the application bell (10, 12), the feeding assembly (70) in its turn comprising:

pump means (516, 418, 531, etc., paragraphs 54, 98, 523, 552) for realizing, in the treatment chamber, an environment having a pressure (P1) lower than the atmospheric pressure;

a controlling unit (91, par. 375) combined with feeding assembly (70) and programmed to activate the pump means and the delivering means in order to have, in the treatment chamber, an environment having a pressure (P1) lower than the atmospheric pressure, eventually together with light, ultrasound, magnetic field, hair removal, electrostimulation treatment (par. 386, 425).

### SUMMARY OF THE INVENTION

Object of the present invention is to provide a device for treating cutaneous blemishes and dermatologic diseases solving the drawbacks of the devices according to the known art.

It is a further object of the present invention to provide a device for treating cutaneous blemishes and dermatologic diseases that is simple to build and cost effective.

Therefore, the present invention relates to a device for treating cutaneous blemishes and dermatologic diseases according to claim 1.

In particular, the device comprises an application bell shaped to be removably leant on a cutaneous surface to be treated. The application bell, once leant on the cutis, deforms elastically in order to fit to the curvature of the skin, and to delimit and define a treatment chamber.

The bell is made by a material preferably impermeable to air and oxygen.

The treatment device further comprises a feeding assembly operatively connected to the application bell. The feeding assembly comprises pump means for creating, in the treatment chamber, an environment having a pressure lower than the atmospheric pressure, and oxygen delivering means to deliver an oxygen flow in the treatment chamber.

The treatment device further comprises a controlling unit combined with the feeding assembly to activate, selectively and in time sequence, the pump means and the oxygen delivering means.

The controlling unit is programmed to realize a working cycle according to which is achieved in the treatment chamber, in time sequence, a depressurized environment having a pressure lower than the atmospheric pressure, and subsequently an environment wherein hyperbaric oxygen is delivered until reaching a substantially hyperbaric environment having a pressure almost equal to the atmospheric pressure, or slightly higher than this, in order not to let the outside air entering the chamber.

For the purposes of the present invention by ‘hyperbaric environment’ is meant an environment in the presence of hyperbaric oxygen, i.e. with 95% minimum.

The two steps of depressurization and subsequent administration of oxygen can be reiterated and allow to achieve the following advantages.

Creating an environment substantially under vacuum in the treatment chamber allows to attract, by increasing it, the blood flow just under the skin of the patient in the treatment area. Instead, the subsequent oxygen delivering step allows the oxygenation of the superficially attracted blood and, therefore, the natural cell regeneration thereby aiding their healing—in case of diseases—or the aesthetic improvement, this also thanks to the combined use of any active principles.

The alternation of the two steps is making the difference by the working cycle of the device object of document CN 102225224. In fact, the device according to the invention is aimed firstly at making a depression in the treatment chamber—and not to constantly maintain a depression—and subsequently at “saturating” vacuum with hyperbaric oxygen. This in order to superficially attract the blood flow, to allow its oxygenation and aid the natural cell regeneration.

For what concerns US 2004/260210, the device shown in FIG. 14, used to make a depression in the treatment chamber, and the device shown in FIG. 23A, used to supply oxygen to a mask (reference 308) or lymphatic drainage system, are not the same device.

In fact, the device shown in FIG. 14 lacks of oxygen delivering means to deliver an oxygen flow in the treatment chamber, and furthermore the controlling unit is not programmed to activate, selectively and alternately, the pump means and the delivering means in order to have, in the treatment chamber, an environment having a pressure lower than the atmospheric pressure and a hyperbaric environment having a pressure substantially equal to the atmospheric pressure or slightly higher, alternately.

Instead, the device shown in FIG. 23A doesn't provide for the application of depression on the cutis, but rather the application of oxygen only.

In essence US 2004/260210 describes two treatment devices, each having its own function, and not a single device including the two functions of creating a depression localized on the cutis of the patient and creating a hyperbaric environment in the same area, alternately over time. Also the three claims 35, 44 and 45 in US 2004/260210 don't provide for the alternate combination over time of the application of vacuum and oxygen by a single device on the cutis of the patient.

Actually, US 2004/260210 describes the use of oxygen regarding the device shown in FIGS. 23A and 23B; with reference to this, see the description at paragraphs 491 to 499. It is true that paragraph 498 specifies “Alternatively a spout 12, for example, as taught in conjunction with FIG. 2A, hereinabove, may be used for oxygen therapy, in place of mask 308”, but read in the context of this paragraph, it simply states that instead of the mask 308 it is possible to use a mouthpiece, as that shown in FIG. 2A and denoted by numeral 12, to let the oxygen arrive to the mouth of the patient.

Paragraphs 456 and 468 make reference to the possibility of using air (not oxygen), but also in this case do not relate to an application alternate with the depression localized on the cutis. Instead, the paragraphs refer to hair or nail treatment, with air flows alternated to depressurizations.

The same considerations also apply to the embodiment described in FIGS. 31A-32B (par. 552-559). Herein a single device is provided that could supply an air flow (not oxygen) and suck in order to create vacuum during the treatment of the face and body, but the possibility to alternate these functions at the cutis level, is not described. Conversely, paragraph 559 clarifies that only one of the two functions is used, depending on the treatment to be carried out.

Among other things, US 2004/260210 does not suggest to integrate the two devices and subject them to a single controlling unit in order to alternate vacuum to the application of oxygen.

Preferably, the device comprises a box-shaped body which defines a housing seat. In the housing seat, pump means and oxygen delivering means, in addition to the controlling unit, are lodged.

The controlling unit, in its turn, comprises a user interface. Preferably, the user interface comprises a display and selecting means to program said controlling unit and vary the working parameters of the device.

The delivering means of the oxygen flow comprise an oxygen concentrator connected by a feeding duct to the application bell. Preferably, the oxygen is hyperbaric oxygen with 95% minimum concentration.

Alternatively, the device can be connected to an oxygen system remote with respect to the treating device. For example, it is an outer oxygen cylinder or centralized oxygen circuit. Also the oxygen concentrator can be a remote device or integrated to the device itself.

Advantageously, the pump means comprise a vacuum pump and a suction duct connected to the application bell.

In a preferred embodiment, the oxygen feeding duct and the suction duct converge in only one branch that provides for at least one connecting plug for the removable connection of a connecting duct extending between the feeding assembly and the bell. The connecting duct plays the role of suctioning and feeding terminal duct in the respective depressurization and oxygen delivering steps provided in the working cycle described above.

Advantageously, a plurality of connecting plugs for the respective application bells, are provided. Therefore, it is possible to contemporaneously carry out the treatment by a unique device in different interest areas with, contemporaneously or alternately active, application bells.

Preferably, the application bell is made of deformable material, in particular a plastic material such as rubber, silicone, elastomers or similar. The bell creates a tight connection with the cutaneous surface.

Advantageously, the bell is equipped with a connecting belt, for example elastic with Velcro closure, in order to better adhere to the patient during the treatment.

Preferably, the bell comprises a flexible perimetrical edge, in particular a rounded edge aiding the adhesion on the cutaneous surface of the patient; this furthermore favors the tightness against the fluid.

Preferably, the pump means are sized to get a negative pressure in the treatment chamber comprised between 20 mmHg and 200 mmHg.

Advantageously, the working cycle operated by the controlling unit provides for, in time sequence, a vacuum time comprised between 0.1 minute and 1 minute and an oxygen delivering time comprised between 0.1 minute and 5 minutes.

Preferably, the working cycle operated by the controlling unit provides for a vacuum time comprised between 5 and 15 seconds and an oxygen delivering time of about 15 seconds.

In an alternative embodiment of the device, a box-shaped body having compact size which defines the feeding assembly and preferably lodges the pump means, the delivering means in addition to the controlling unit themselves also conveniently sized, is provided. The application bell is operatively and directly integrated on the box-shaped body; the so-configured device is able to be portably grasped by an operator with the purpose of proceeding to the treatment of the patient.

### LIST OF FIGURES

Further characteristics and advantages of the invention will be more evident from a review of the following specification of a preferred, but not exclusive, embodiment, shown for illustration purposes only and without limitation, with the aid of the attached drawings, in which:

FIG. 1 shows a perspective view of a treatment device according to the invention in a first working step;

FIG. 1A shows a perspective view of the application bell of the treatment device of FIG. 1;

FIG. 2 shows a perspective view of a treatment device of FIG. 1 in a second working step;

FIG. 3 shows a schematic view of the pneumatic/electrical circuit of the treatment device, according to the present invention.

### DETAILED DESCRIPTION OF THE INVENTION

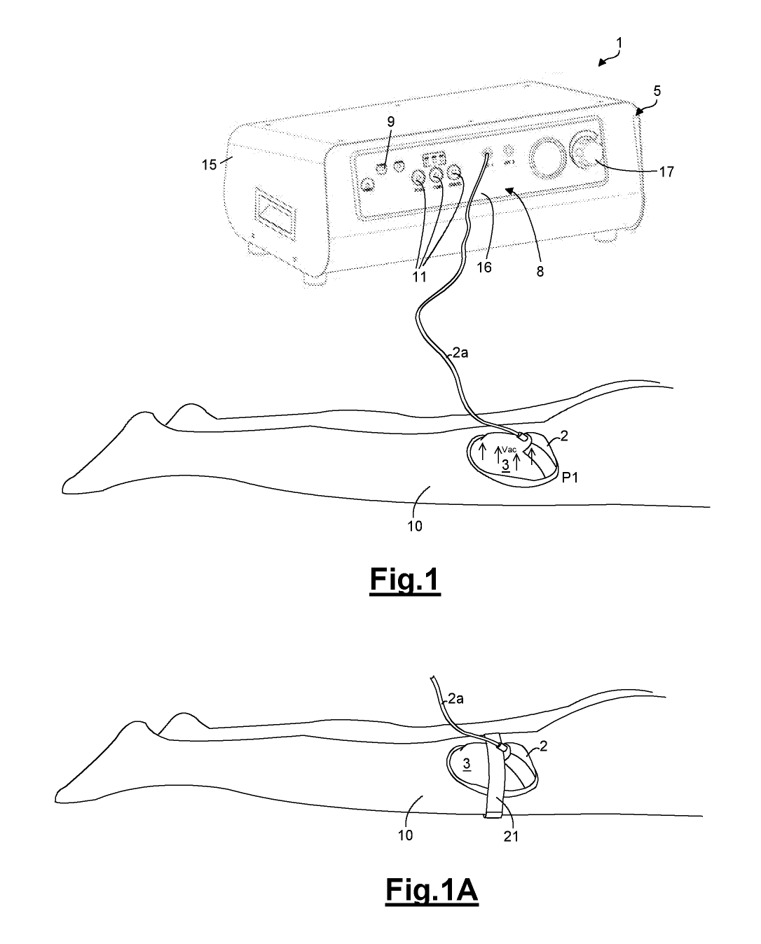

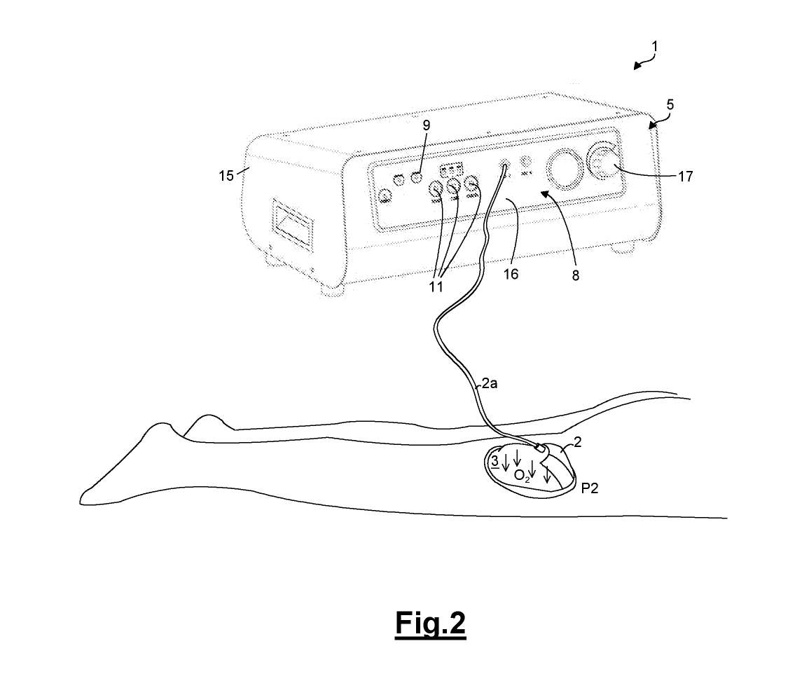

With reference to FIGS. 1 and 2 a device 1 for treating cutaneous blemishes and dermatologic diseases, according to the invention, is schematically shown. In particular, the device 1 comprises an application bell 2 shaped to be removably leant on a cutaneous surface 10 to be treated of a patient 10. The application bell 2 circumscribes on the cutaneous surface 10 a treatment chamber 3.

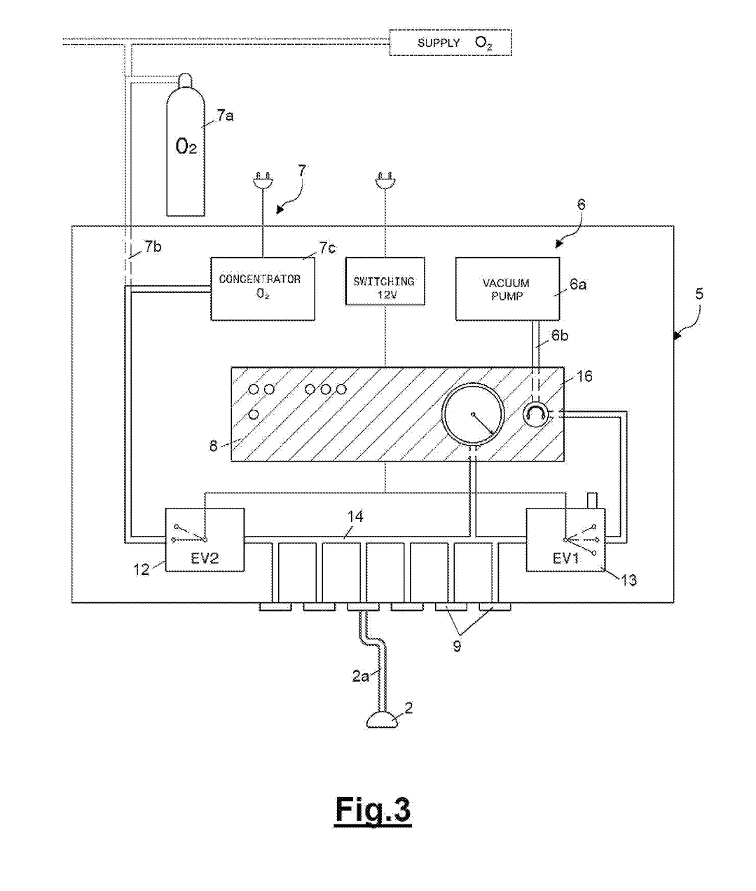

The application bell 2 is operatively combined with a feeding assembly 5 comprising—as shown in the schematic FIG. 3—pump means 6 for realizing, in the treatment chamber 3, an environment having a pressure lower than the atmospheric pressure (FIG. 1), and oxygen delivering means 7 to deliver an oxygen flow in the treatment chamber 3 (FIG. 2) following the creation of the depression.

As described below, the feeding assembly 5 is operated by a controlling unit 8 to selectively activate, in time sequence, the pump means 6 and the delivering means 7.

According to a working cycle of the device 1 of the invention, the controlling unit 8 is programmed in order to have, in time sequence in the treatment chamber 3, an environment under vacuum with a pressure lower than the atmospheric pressure, and subsequently a hyperbaric oxygen environment that brings back the pressure in the treatment chamber 3 to a pressure substantially equal to the atmospheric pressure or slightly higher.

The working cycle operated by the controlling unit 8 provides for a depression or vacuum time comprised between 0.1 minute and 1 minute and an oxygen delivering time comprised between 0.1 minute and 5 minutes.

Preferably, the working cycle operated by the controlling unit 8 provides for a depression or vacuum time comprised between 5 and 15 seconds and an oxygen delivering time of about 15 seconds.

Preferably, the device comprises a box-shaped body 15 which defines a housing seat. In the housing seat, pump means 6 and oxygen delivering means 7, in addition to the controlling unit 8, are preferably lodged.

The controlling unit 8, in its turn, comprises a user interface 16. Preferably, the user interface 16 comprises a display and selecting means 11 and 17 to program the working parameters of the device.

The delivering means 7 of the oxygen flow, that can be located outside and connected to the device or lodged, advantageously comprise an oxygen concentrator 7c fluidically connected by a feeding duct 7b to the application bell 2. The oxygen concentrator 7c is able to produce oxygen withdrawing outside air.

Alternatively, the delivering means 7, still as schematically shown in FIG. 3, comprise an oxygen cylinder 7a or a main oxygen supply 7d.

The flow incoming to the bell 2 is activated by a first electro-valve 12. The oxygen is preferably hyperbaric 95% oxygen. Alternatively, the oxygen cylinder 7a is remote with respect to the treating device. For example, it is an outer oxygen cylinder or centralized oxygen circuit.

Advantageously, the pump means 6 comprise a vacuum pump 6a and a suction duct 6b fluidically connected to the application bell 2. Also in such a case the vacuum pump 6a is combined with a second electro-valve 13 to operate the suction step in the application bell 2.

The oxygen feeding duct 7b and the suction duct 6b converge in only one branch 14 that provides for at least one connecting plug 9, in particular a plurality of plugs 9, for a connecting duct 2a extending between the feeding assembly 5 and the bell 2. The connecting duct 2a plays the role of suction duct and feeding duct in the respective steps of creating vacuum and delivering oxygen, which are provided in the working cycle.

According to further constructive aspects, the application bell 2 is made of deformable material, in particular a plastic material such as rubber, silicone, elastomers or similar, in order to create a tight connection with the cutaneous surface 10 of the patient. Preferably, the bell 2 comprises a flexible perimetrical edge, in particular a rounded edge aiding the adhesion on the cutaneous surface 10 of the patient. The bell 2 can be equipped with a belt 21, preferably elastic with Velcro closure, in order to better adhere to the patient during the treatment (FIG. 1A)

The pump means 6 are sized to get a negative pressure in the treatment chamber 3 comprised between 20 mmHg and 200 mmHg.

In an alternative embodiment of the device, not shown in figures, a box-shaped body having compact size is provided and defines—in the above described variation—the feeding assembly 5 and preferably integrates the pump means 6, the delivering means 7 and the controlling unit 8, also themselves conveniently sized. The application bell 2 is operatively and directly lodged on the box-shaped body. The bell is not therefore in remote position with respect to the feeding assembly 5. Therefore, the box-shaped body is able to be portably grasped by an operator with the purpose of proceeding to the treatment of the patient.

1. A device (1), for treating cutaneous blemishes and dermatologic diseases, comprising:
an application bell (2) shaped to be removably leant on a cutaneous surface (10) to be treated and with which the application bell (2) defines a treatment chamber (3);
a feeding assembly (5) operatively connected to the application bell (2), the feeding assembly (5) in its turn comprising:
a pump (6), which realizes, in the treatment chamber (3), an environment having a pressure (P1) lower than atmospheric pressure;
an oxygen delivering device (7) configured to deliver an oxygen flow into the treatment chamber (3);
a controlling unit (8) combined with said feeding assembly (5) and programmed to activate selectively and alternately the pump (6) and the oxygen delivering device (7) in order to have, in the treatment chamber (3), an environment having a pressure (P1) lower than the atmospheric pressure and a hyperbaric environment having a pressure (P2) substantially equal to or slightly higher than atmospheric pressure.

2. The device according to claim 1, wherein the controlling unit (8) is programmed for reiterating over time the activation of the pump (6) and oxygen delivering device (7) of the feeding assembly (5).

3. The device according to claim 1, wherein a box-shaped body (15) is provided and defines a housing seat, and wherein in the housing seat the pump (6) and the oxygen delivering device (7)—that define the feeding assembly (5)—in addition to the controlling unit (8), are lodged, and wherein the box-shaped body (15) is remote with respect to the application bell (2).

4. The device according to claim 1, wherein a box-shaped body is provided having compact size, which defines a housing seat, and wherein in the housing seat the pump (6) and the oxygen delivering device (7) in addition to the controlling unit (8) are lodged, and wherein on the box-shaped body having compact size the application bell (2) is integrated, said box-shaped body being able to be portably grasped by an operator for carrying out the treatment.

5. The device according to claim 3, wherein the controlling unit (8) comprises, in its turn, a user interface (16), and wherein the user interface (16) comprises a display and selecting interface (11) to vary the working parameters of the device.

6. The device according to claim 1, wherein the oxygen delivering device (7) comprises: an oxygen concentrator (7c) or a remote oxygen cylinder; or an outer distribution circuit (7a) and a feeding duct (7b) connected to the application bell (2).

7. The device according to claim 6, wherein the pump (6) comprises a vacuum pump (6a) and a suction duct (6b) fluidically connected to the application bell (2), and wherein the pump (6) is sized to provide a negative pressure in the treatment chamber (3) comprised between 20 mmHg and 200 mmHg.

8. The device according to claim 7, wherein the oxygen feeding duct (7b) and the suction duct (6b) converge in only one branch (14) that provides for a plurality of plugs (9), for the removable connection of a connecting duct (2a) extending between the feeding assembly (5) and the bell (2).

9. The device according to claim 1, wherein the application bell (2) is made of deformable material selected from the group consisting of: a plastic material; rubber, silicone, elastomers or combinations thereof, and wherein the bell comprises a flexible perimetrical edge, a self-adapting rounded edge aiding adhesion and tightness on the cutaneous surface (10) of the patient.

10. The device according to claim 1, wherein a working cycle operated by the controlling unit (8) provides—in time sequence—a vacuum time comprised between 0.1 minute and 1 minute, an oxygen delivering time comprised between 0.1 minute and 5 minutes.

11. The device according to claim 1, wherein a working cycle operated by the controlling unit (8) provides—in time sequence—a vacuum time comprised between 5 and 15 seconds and the oxygen delivering time is about 15 seconds.
